(19) United States
(12) Patent Application Publication
(10) Pub. No.: US 2015/0344897 A1
(43) Pub. Date: Dec. 3, 2015

(54) MONOCOTYLEDON TRANSGENIC METHOD FOR INVADING GROWING POINTS OF SEED BUDS MINIMALLY AND FULLY

(71) Applicants: Haibo Wang, Shijiazhuang (CN); Fushuang Dong, Shijiazhuang (CN); Mengyu Lv, Shijiazhuang (CN); Yanmin Zhang, Shijiazhuang (CN); Zhiheng Ren, Shijiazhuang (CN); Fan Yang, Shijiazhuang (CN); Xinchao Liang, Shijiazhuang (CN); Wenbo Zuo, Shijiazhuang (CN); Xueping Shi, Shijiazhuang (CN); Huanhuan Zhang, Shijiazhuang (CN); Yiping Gao, Shijiazhuang (CN); He Zhao, Shijiazhuang (CN); Xian Xu, Shijiazhuang (CN); Guozhong Sun, Shijiazhuang (CN); Jianfang Chai, Shijiazhuang (CN); Yongwei Liu, Shijiazhuang (CN); Jinyong Zhu, Shijiazhuang (CN); Qiufen Han, Shijiazhuang (CN); Qiang Zhang, Shijiazhuang (CN); Huijie Ma, Shijiazhuang (CN); Zhanwu Wang, Shijiazhuang (CN); Junfeng Guan, Shijiazhuang (CN)

(72) Inventors: Haibo Wang, Shijiazhuang (CN); Fushuang Dong, Shijiazhuang (CN); Mengyu Lv, Shijiazhuang (CN); Yanmin Zhang, Shijiazhuang (CN); Zhiheng Ren, Shijiazhuang (CN); Fan Yang, Shijiazhuang (CN); Xinchao Liang, Shijiazhuang (CN); Wenbo Zuo, Shijiazhuang (CN); Xueping Shi, Shijiazhuang (CN); Huanhuan Zhang, Shijiazhuang (CN); Yiping Gao, Shijiazhuang (CN); He Zhao, Shijiazhuang (CN); Xian Xu, Shijiazhuang (CN); Guozhong Sun, Shijiazhuang (CN); Jianfang Chai, Shijiazhuang (CN); Yongwei Liu, Shijiazhuang (CN); Jinyong Zhu, Shijiazhuang (CN); Qiufen Han, Shijiazhuang (CN); Qiang Zhang, Shijiazhuang (CN); Huijie Ma, Shijiazhuang (CN); Zhanwu Wang, Shijiazhuang (CN); Junfeng Guan, Shijiazhuang (CN)

(73) Assignee: HEBEI ACADEMY OF AGRICULTURE AND FORESTRY SCIENCES, INSTITUTE OF GENETICS AND PHYSIOLOGY, Shijiazhuang, Hebei (CN)

(21) Appl. No.: 14/422,661

(22) PCT Filed: Sep. 14, 2012
(86) PCT No.: PCT/CN2012/001265

§ 371 Date: Feb. 19, 2015

(30) Foreign Application Priority Data
Aug. 22, 2012 (CN) 201210300322.9

(51) Int. Cl. C12N 15/82

(52) U.S. Cl. CPC C12N 15/8207; C12N 15/8205

(57) ABSTRACT

The present invention is a method of shoot apical meristem transformation for monocot plant via sufficient and micro wounding (SMW). The technical process includes: expose the apical meristem by removing the coleoptile away when the shoot grows to 0.2-2 cm after 1-2 days of seed germination; make sufficient and micro wounding transformation to the apical meristem by stabbing and brushing for 2-3 times using the SMW brush having 100-5000 bristles which is 4-20 μm in diameter for each one and 0.5-3 mm in exposed length, and dipped with the Agrobacterium tumefaciens containing binary vector harboring exogenous genes; develop the treated meristems directly to normal plants after co-cultivation; promote the plants to develop big spikes and set more seeds; harvest the seeds of T₀ plants separately; detect and identify the transformation results in T₁ generation which is bred from each individual T₀ plant. The advantages of the invention are independent of tissue culture, unlimited in genotype, unnecessary to carry resistant marker, simple and large scale to perform, and applicable to all monocot plants which can set seeds. The transformation efficiencies for wheat, rice and maize using this method are 49%, 66.3%, and 100%, respectively.

### TECHNICAL FIELD

The present invention is a method of shoot apical meristem transformation for monocot plant via sufficient and micro wounding (SMW), applicable to all monocot plants which can set seeds.

### BACKGROUND ART

Transferring gene via A. tumefaciens is the most acceptable approach among a plurality of transformation methods for plants. It has several significant advantages including high fertility for transgenic plant, single or low copy number for exogenous gene integration, and suitable for transferring long fragment of DNA, etc. However, the conventional transformation technology via A. tumefaciens is always dependent on tissue culture which is limited in genotype, complicated to perform, necessary to carry resistant marker, low efficiency, and poor repeatability, especially in monocot plants, for example wheat, rice and maize, which are strongly limited in genotype, thus the development and application of this method are seriously restricted.

The shoot apical meristem of seed is the original cells which can develop and differentiate to the whole reproduction and the most shoot organs. The apical meristem of seed in monocot plant is one of the most ideal objects for transformation, due to its strong ability in recovery and compensation for development, which can grow to normal seedling after the coleoptile and the little leaves were removed away, and even being suffered serious wounding.

Some reports and patents have mentioned the advantages to use shoot apical meristem as transformation object. However, practical and efficient approach has not been established, due to the poor understanding on characteristics of shoot apical meristem. For example: the initial time is too late for transformation, which is resulted in low coverage for the apical meristem cells; the transformation of apical meristem with no wounding, or too heavy wounding, or insufficient wounding lead to low efficiency; the resistant screen in T₀ plants may eliminate some real transformed chimeras or retain some false ones.

Chinese patent KK a modified transformation method for wheat apical meristem via A. tumefaciens) (NO: 200410075773.2) has mentioned the main steps for transformation: (1) vernalize the seeds under 4° C. for 20-30 days after germination; (2) activate and resuspend the A. tumefaciens harboring exogenous gene; (3) take the suitable size of seedling, to expose or make wounding to the apical meristem by sideling cutting off the above part; (4) drip the infection solution of A. tumefaciens harboring exogenous gene to the oblique section of the rest part of seedling; (5) redevelop the rest part to a completed seedling and transplant it into soil, and then conduct chemical screen in the plants and their progeny; (6) perform molecular identification for the resistant plants and their progeny. Said suitable size of seedling is 2-4 cm in length.

The main problems of this patent were below:

(1) The period is too late to perform transformation after such long time of vernalization, which would result in rare opportunity for effective treatment. The operation not only provides very little wounding for A. tumefaciens transformation, but also makes too heavy damage to the apical meristem.

(2) The method is quite difficult to manage, especially hardly to cut off the above part sideling of the seedling properly, and resulted in low operation efficiency.

Some studies have reported that the apical meristem can be stabbed using needles. However, most of the used needle is usually too thick to make wounding [African Journal for Biotechnology, 2011, 10(5): 740-750], even its diameter is up to 710 μm [Journal of bioscience and bioengineering, 2005, 100(4): 391-397; 2006, 102(3):162-170]. Using such kind of needles to stab the meristem would result in heavy damage to it, but little desirable wounding for A. tumefaciens infection. In some publications, the wounding treatment is only to scratch the apical meristem randomly using a blade, which is hard to meet the requirement for A. tumefaciens transformation and rarely to obtain good results. In many other reports, transformation via A. tumefaciens is conducted without any wounding treatment to apical meristem, which is difficult to keep the effect of manipulation.

In summary, many of the reports and patents which use the apical meristem as object for transformation are still dependent on in vitro culture, and the resistant screen recommended is not reasonable.

### SUMMARY OF THE INVENTION

The demand of this invention is to establish a novel method for shoot apical meristem transformation of monocot plant via sufficient and micro wounding (SMW), which are independent of tissue culture, unlimited in genotype, unnecessary to carry resistant marker, simple and large scale to perform, stable in transformation result, practical, and low cost.

Technical scheme for this invention is below:

A method of shoot apical meristem transformation for monocot plant via sufficient and micro wounding, comprising the steps of:

(1) Preparation of In Vivo Meristems and Infection Solution

Select healthy and complete seeds of the objective crops, remove the chaff or the husk away, wash the seeds clean and soak them in water at 25° C. for 7-10 hours. After routine sterilization, rinse the seeds in sterilized water several times and place them on two layers of autoclaved absorbent tissue in a Petri dish (Φ90 mm). Drip sterilized water with an amount just to keep the absorbent tissue wet, and then germinate the seeds at 28° C. in dark for 1-2 days. Said in vivo meristem for transformation is the shoot growing point of the seed treated in this way.

Screen single colony of A. tumefaciens containing binary vector harboring exogenous genes, and inoculate it into LB medium containing 50 mg/L kanamycin and 40 mg/L rifampicin and grow to OD₆₀₀=0.5-0.6 at 28° C. on shaker with 220 rpm in dark. Prepare the A. tumefaciens infection solution by centrifugating the culture at 4000 rpm for 5 min and re-suspending it in the base buffer of 1/5-1/2 volume as the original. Said base buffer contains 1/10 MS medium complemented with 100 μM AS, 100 mg/L F68, 400 mg/L MES, 10 g/L glucose and 40 g/L maltose, pH 5.6.

(2) Expose the Shoot Apical Meristem and Transform it via SMW

\textcircled{1} Initial Time Manage:

the transformation should be conducted as early as possible, the suitable time is when the shoot has grown to 0.2-2 cm for little grain plant, and 0.3-1 cm for big grain plant.

Said plants with little grains comprise wheat, rice, millet, broomcorn millet, and sorghum; and said plant with big grain comprises maize.

\textcircled{2} Method to Expose the Shoot Apical Meristem

For the plants in which subterranean stem have not elongated, directly remove the coleoptile and little leaves away from its base using a tweezer; for the plants in which subterranean stem have elongated, cut the coleoptile and little leaves away using a blade just in the above of the light reflection belt between the subterranean stem and the coleoptile, where it is the region of the shoot apical meristem.

\textcircled{3} Transformation Using the SMW Brush

Take the SMW brush dipped with the A. tumefaciens infection solution, stab and brush the apical meristem for 2-3 times. Thereafter, place the seeds with apical meristem up on two layers of dry absorbent tissue in the Petri dish which have been autoclaved together. Each Petri dish can be put in 10-40 seeds.

(3) Co-Cultivation

Drip 0.5-3 mL of sterilized water on the absorbent tissue in Petri dish containing the transformed seeds, and then specially co-culture the in vivo meristems at 25° C. in dark for 3 days with the dish lid covered.

(4) Develop to Seedlings

After co-cultivation, cover the roots with vermiculite in the dish, or transplant the little seedlings to the bowl containing vermiculite.

For the plants which do not require vernalization, grow the little seedlings at 25° C. with a 12-h photoperiod for 7 days, and then transplant them into pot in greenhouse, or directly transplant the young seedlings into pot in greenhouse if they have developed relatively big and cover with plastic film to recover or protect them for 7-10 days.

For the plants which require vernalization, such as winter wheat, grow the little seedlings at 25° C. with a 12-h photoperiod for 7 days, and then transfer them into the 8° C. growth chamber for 20-30 days, the time required depends on the cultivar.

(5) Transplant the Seedlings

Transplant the seedlings into the environmentally controlled greenhouse or farmland, when they are developed enough.

(6) Seedling and Plant Management

Promote the seedlings and plants healthily to develop big spikes and set more seeds with water and nutrition management. For maize with unisexual flowers, protect the ear and tassel with paper bags and then carry out the pollination artificially.

(7) Molecular Detection and Identification

Do not perform the detection, selection and identification in T₀ plants to avoid false results. Harvest the seeds of T₀ plants separately. Perform the molecular detection and identification in T₁ generation. For the plants which have been transformed with exogenous vector harboring resistance gene, screen the resistant plants and then carry out PCR identification; for the plants transformed without resistance gene, carry out PCR identification directly. Perform Southern blot analysis for PCR-positive plants.

The bristles of said SMW brush are made with stainless steel fibers, glass fibers or carbon silicon fibers in micron-grade. One bristle is 4-20 μm in diameter and 0.5-3 mm in exposed length, and one brush contains 100-5000 bristles.

Said SMW brush in which bristle is 8-18 μm in diameter, the bristle is 1-2 mm in exposed length, and each brush contains 100-2000 of bristles.

Said “stab and brush” is mean not only to stab but also to brush on the apical meristem. Said “stab” is to prick the apical meristem vertically with the SMW brush dipped with the A. tumefaciens infection solution to transfer the exogenous genes; and said is to comb the whole apical meristem with the SMW brush dipped with the A. tumefaciens infection solution to transfer the exogenous genes.

Said plants wherein subterranean stem could not elongate comprise wheat and rice; and said plants wherein subterranean stem could elongate comprise maize, millet, broomcorn millet, and sorghum.

Technical principle of the invention is below:

The applicants find some critical issues through a systematic study: (1) Transformation can be conducted when the seeds germinate until the coleoptile can be removed away for wheat, maize, and rice, and the same conclusion is summarized in millet, broomcorn millet, and sorghum. Based on these results, the earliest appropriate period for apical meristem transformation was determined. (2) In order to obtain good transformation result, sufficient and micro wounding to the tender apical meristem is necessary. Thus, the applicants invent a novel instrument for plant transformation, which can make sufficient and micro wounding to apical meristem. So it is called sufficient and micro wounding brush, abbreviated as SMW brush. The brush contains 100-5000 bristles (4-20 μm in diameter for each) which are made of stainless steel fibers, glass fibers or carbon silicon fibers. Good transformation can be obtained using this kind of brush dipped with A. tumefaciens infection solution. (3) Results can be improved by properly controlling the water potential of the seedling after transformation, in order to avoid the cells burst or wilting and to promote the A. tumefaciens close to the meristem cells. (4) After being treated with SMW brush, most of the shoot meristems can develop normally to set seeds and the transformation save time with higher efficiency compared to other methods. (5) The seeds are harvested separately from every T₀ plant, and then to be germinated to seedlings. Molecular identification is performed in T₁ generation. This strategy no longer need resistant screen and can accurately show the status transformed.

Based on these recognitions, the special evaluation index is established for this transformation technique:

The damage rate and the normal seedling rate: after stabbing and brushing, some of the shoot apical meristems are usually damaged and cannot develop normally. The damage rate is the percentage of the treated meristems which cannot further develop. Conversely, normal seeding rate is the percentage of the treated meristems which can develop normally.

$$The damage rate = \frac{number of treated meristems - number of normal seedlings}{number of treated meristems} \times 100 \%$$

$$The normal seedling rate = \frac{number of normal seedlings}{number of treated meristems} \times 100 \%$$

The transformation rate is the percentage of T₀ plants which set positive seeds proved in T₁ generation.

$$The transformation rate = \frac{number of {T}_{0} plants setting positive seeds}{number of {T}_{0} plants setting seeds} \times 100 \%$$

The transformation degree is the percentage of the positive seeds for each individual T₀ plant. This index reflects the transformation degree and status for an apical meristem.

$$The transformation degree = \frac{number of positive seeds of a {T}_{0} plant}{number of all seeds of the {T}_{0} plant} \times 100 \%$$

The advantages of the invention for A. tumefaciens-mediated transformation of monocot plants are no longer to need tissue culture and carry resistant marker, unlimited in genotype, high transformation efficiency, easy to perform in large scale, stable, practical, low cost, and applicable to all monocot plants which can set seeds.

### BRIEF DESCRIPTION OF THE FIGURES

FIG. 1 The structure of the SMW brush. A: The SMW brush with bristles made of stainless steel fibers; B: The SMW brush with bristles made of glass fibers; C: The SMW brush with bristles made of carbon silicon fibers; a: The SMW brush has 4000 bristles which are 8 μm in diameter for each and made of stainless steel fibers; b: The SMW brush has 200 bristles which are 4 μm in diameter for each and made of stainless steel fibers; c: The SMW brush has 200 bristles which are 16 μm in diameter for each and made of stainless steel fibers.

FIG. 2 The longitudinal structure of the apical meristems of gramineous plants through micro observation. A: The longitudinal structure of the apical meristem of wheat through micro observation; B: The longitudinal structure of the apical meristem of rice through micro observation; C: The longitudinal structure of the apical meristem of maize through micro observation.

FIG. 3 Album of sufficient and micro wounding transformation for apical meristem of wheat, a typical monocot plant. a: Removing the coleoptile away to expose the apical meristem; b: Microscope view for apical meristem (framed by the circle); c: The head of SMW brush; d: Transformation via the SMW brush; e: The objects have been treated with SMW brush for transformation; f: Shoot development after transformation; g: T₀ seedlings; h: T₀ plants; i: The seeds harvested from T₀ plants (the origin of Tᵢ); j: Resistant screen for T₁ plants (The albino was negative, and non albino was positive. Some leaves of resistant plants were collected for molecular identification); k: PCR results.

FIG. 4 Album of all kinds of the transforms and their progenies via sufficient and micro wounding transformation. a: The environmentally controlled greenhouse for transgenic plants; b: T₁ seedlings of transgenic wheat; c: T₂ plants of transgenic rice (set seeds); d: T₂ plants of transgenic maize; e: T₀ plants of millet after transformation; f: T₀ plants of sorghum after transformation.

FIG. 5 PCR results of gus gene in T₁ plants of wheat. Lane 1 to lane 21 are the detected samples, lane 22 is the blank control, lane 23 is the negative control, lane 24 is the positive control, and lane 25 is DNA marker DL2000.

FIG. 6 Southern blot analysis based on PCR product of gus gene for wheat. Lane ‘CK’ is the positive control, lane 1 is the blank control, lane 2 is the negative control, and lane 3 to lane 11 are the detested samples.

FIG. 7 Southern blot analysis for wheat genomes. Lane 1 to lane 6 are the detected samples, lane 7 is the blank control, lane 8 is the negative control, and lane ‘M’ is the marker.

FIG. 8 PCR results of bar gene in T₁ plants of rice. Lane 1 is the DNA marker DL2000, lane 2 is the positive control, lane 3 is the blank control, lane 4 is the negative control, and lane 5 to lane 25 are the detected samples.

FIG. 9 PCR results of bar gene in T₁ plants of maize. Lane ‘M’ is the DNA marker DL2000, lane ‘CK’ is the positive control, lane ‘CK⁻’ is the negative control, and lane 1 to lane 16 are the detected samples.

FIG. 10 Southern blot analysis in T₁ plants of maize. Lane ‘M’ is the DNA marker, lane 1-6 are the detected samples, lane 7 is the blank control, lane 8 is the negative control, lane 9 is the vector, and lane 10 is the PCR product.

FIG. 11 Southern blot analysis for T₂ plants of maize from the 26th batch of transformation. It is indicated that the badh gene is double copy of integration.

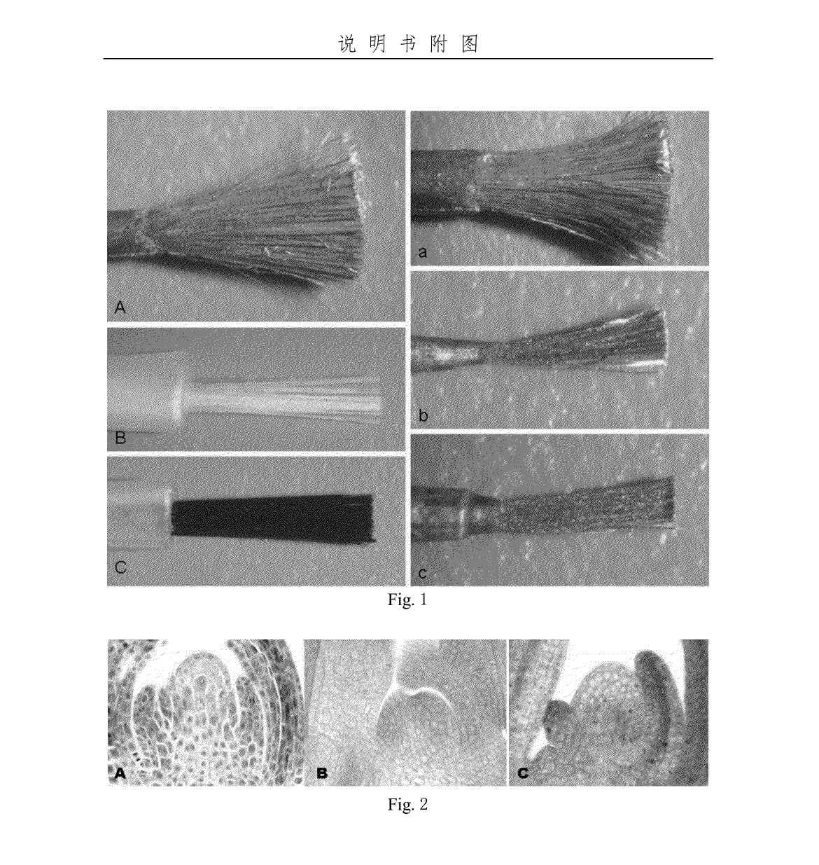

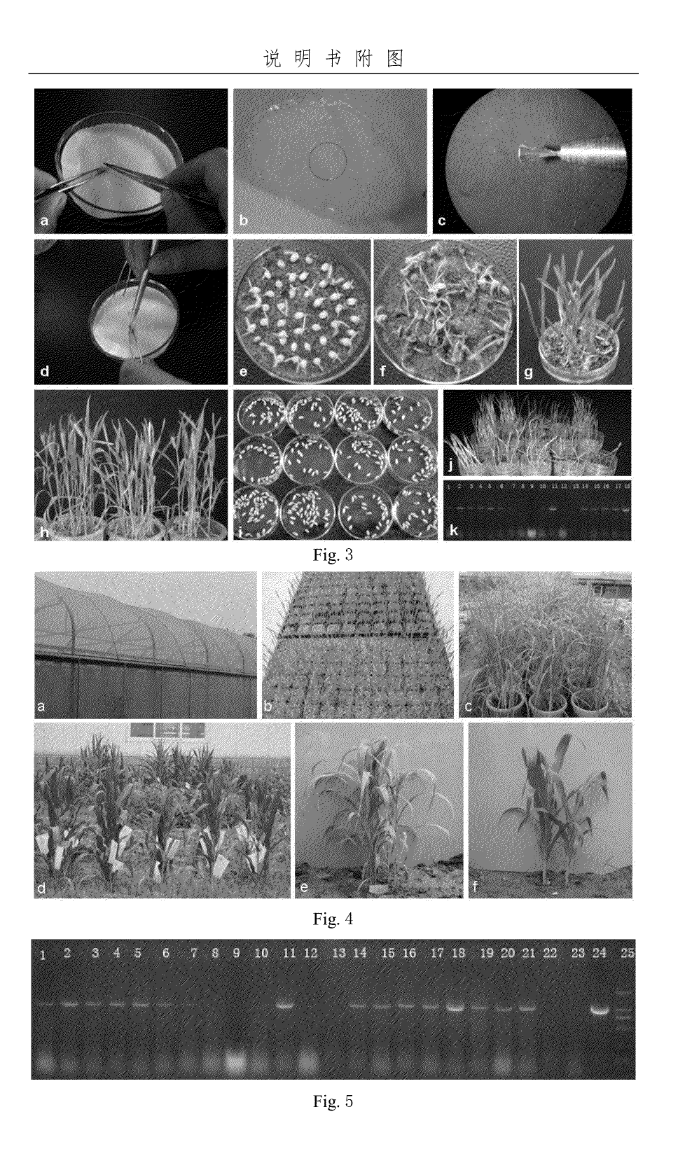

### DETAILED DESCRIPTION OF THE PREFERRED EMBODIMENT

### Embodiment 1

The transformation for apical meristem of winter wheat using the SMW brush

(1) Materials and Methods

Wheat cultivar: Shi 4185.

A. tumefaciens strain: EHA105.

The exogenous genes: gus gene and npt-II gene, constructed in vector pCAMBIA2201.

Single colony of A. tumefaciens was screened and inoculated into 50 mL of LB medium containing 50 mg/L kanamycin and 40 mg/L rifampicin, and grew to OD₆₀₀=0.6 at 28° C. on shaker with 220 rpm. The A. tumefaciens infection solution was obtained by centrifugating the culture at 4000 rpm for 5 min and re-suspended in the base buffer (1/2 volume of the original) containing 1/10 MS medium complemented with 100 μM AS, 100 mg/L F68, 400 mg/L MES, 10 g/L glucose and 40 g/L maltose, pH 5.6.

90 full and healthy seeds were soaked in water at 25 ° C. for 7 hours and sterilized routinely. The seeds were rinsed several times with sterilized water and placed in the autoclaved Petri dish (Φ9 cm) with two layers of absorbent tissue. Sterilized water was dripped with an appropriate amount just to keep the tissue wet. The Petri dish was placed at 28° C. in dark for one day. 86 seeds germinated normally and the coleoptile grew to 0.2-0.5 cm. Then the coleoptile was removed away using a tweezer to expose the apical meristem. The exposed meristem was stabbed and brushed for 2-3 times using SMW brush (5000 bristles which are 12 μm in diameter for each, 3 mm in exposed length) dipped with the A. tumefaciens infection solution. Thereafter, the objects were placed in the autoclaved Petri dish with the exposed side up. Each Petri dish contained 40 seeds and two layers of tissue were wetted with 0.5 mL of sterilized water. The Petri dish was covered with lid and placed at 25° C. in dark for 3 days, and then the objects were planted to the bowl containing wet vermiculite at 25° C. under a 12-h photoperiod for 7 days. Then the seedlings were transferred to the 8° C. growth chamber for 20 days of vernalization. Afterwards the seedlings were transplanted into the environmentally controlled greenhouse. The seeds were harvested from individual plant separately.

According to each T₀ plant, The seeds were harvested and soaked separately in 75 mg/L kanamycin solution (the amount is 1 seed can share 1 mL) for about 1 day at 25° C. until the seeds began to germinate, and then the seeds were sowed in the bowl containing wet vermiculite and placed at 25° C. under a 12-h photoperiod for 7 days. The green plants were counted and traced to their T₀ plants. Some parts of the leaves were collected from every green plant for detection and the green plants were all transferred to the 8° C. growth chamber under a 12-h photoperiod for 25 days of vernalization.

Total genomic DNA was extracted individually from some leaves of every plant. PCR was conducted with gus fragment primers: forward 5′-CAA CGA ACT GAA CTG GCA G-3′ and reverse 5′-CAT CAC CAC GCT TGG GTG-3′. Based on PCR results, the transformation degree was expressed as the percentage of the positive seeds for each individual T₀ plant; and the transformation rate was expressed as the percentage of T₀ plants which set positive seeds proved in T₁ generation. PCR-Southern blot analysis was performed with the PCR product mph tied from the new genomic DNA of positive plants selected randomly. Southern blot was conducted with PCR-Southern blot positive plants (performed by Beijing Meilaibo Medical Technology Co. Ltd.).

(2) Results

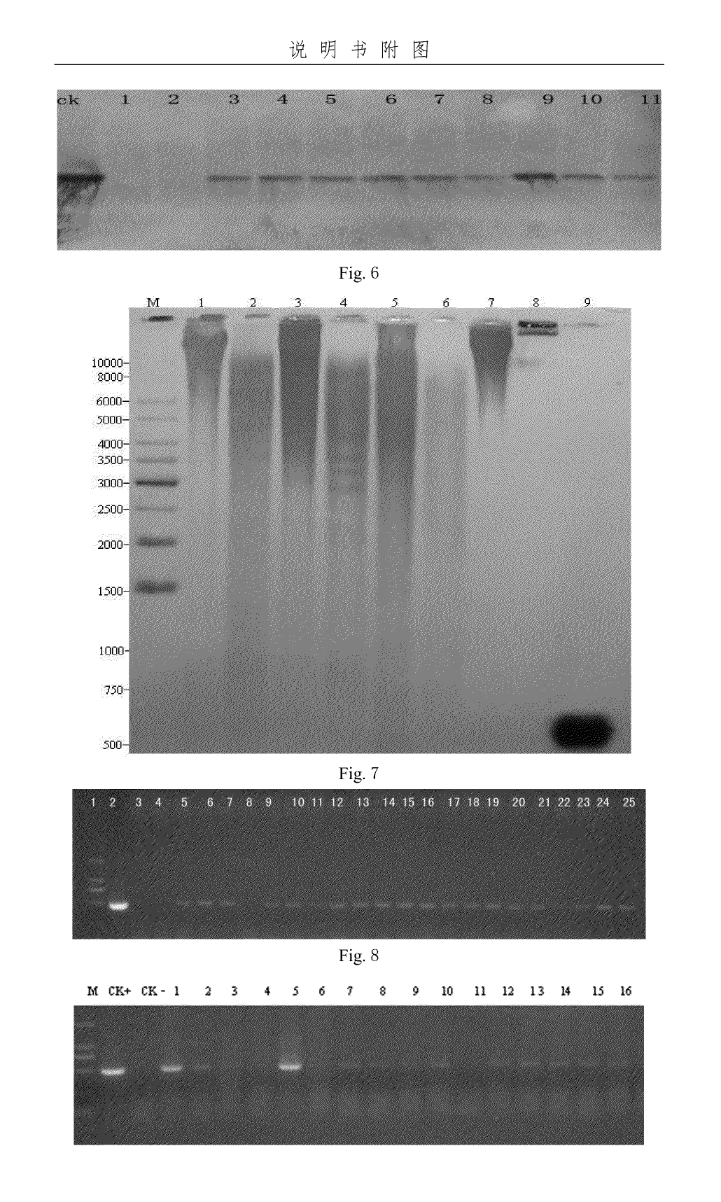

86 out of 90 seeds germinated normally and they were used for transformation. 62 of the treated objects developed to seedlings (the damage rate was 28%) and 53 of them grew to set seeds. The seeds were harvested separately according to the individual T₀ plant, and the screen was performed with kanamycin solution treatment. 373 T₁ green seedlings were obtained and they were from 43 T₀ plants. The resistant rate was 81.1% (43/53×100%). Based on kana-resistant selection, PCR analysis was carried out. Results showed that 111 of the seedlings were positive and they were from 26 T₀ plants. The transformation rate was 49% (26/53×100%) and the transformation degree was 2.6% (1/39 X 100%, 1 out of 39 seeds from a T₀ plant was positive) to 37.1% (23/62×100%, 23 out of 62 seeds from a T₀ plant were positive). PCR-Southern blot showed the same band as the frequent from the vector, which based on 9 PCR-positive plants selected randomly (FIG. 6). Southern blot analysis with the genomic DNA of this kind of plants also showed positive results (FIG. 7). It indicated that the exogenous gene has been integrated into the wheat genome.

### Embodiment 2

### The Transformation for Apical Meristem of Different Genotypes of Wheat Using SMW Brush

(1) Materials and Methods

Wheat cultivars: Jinhe 9123, Chinese Spring, and Bobwhite.

A. tumefaciens strain: C58C1.

The exogenous genes: gus gene and npt-II gene, constructed in vector pCAMBIA2201.

Single colony of A. tumefaciens was screened and inoculated into 50 mL of LB medium containing 50 mg/L kanamycin and 40 mg/L rifampicin, and grew to OD₆₀₀=0.5 at 28° C. on shaker with 220 rpm. The A. tumefaciens infection solution was obtained by centrifugating the culture at 4000 rpm for 5 min and re-suspended in the base buffer (1/5 volume of the original) containing 1/10 MS medium complemented with 100 μM AS, 100 mg/L F68, 400 mg/L MES, 10 g/L glucose and 40 g/L maltose, pH 5.6.

The full and complete seeds of three cultivars were soaked in water at 25° C. for 10 hours and sterilized routinely. The sterilized seeds were rinsed several times with sterilized water and placed in the autoclaved Petri dish (Φ9 cm) with two layers of absorbent tissue. Appropriate quantities of sterilized water were dripped to keep the tissue wet. The Petri dish was placed at 28° C. in dark for one day. The coleoptile which grew to 0.2-0.4 cm was removed away to expose the apical meristem using a tweezer. The exposed meristem was stabbed and brushed for 2-3 times using SMW brush (100 bristles which are 18 μm in diameter for each, 3 mm in exposed length) dipped with the A. tumefaciens infection solution. Thereafter, the treated objects were placed in the autoclaved Petri dish (the inner tissue soaked with 0.5 mL of sterilized water) with the exposed side up. The Petri dish was covered with lid and placed at 25° C. in dark for 3 days, and then the little seedlings were planted to the bowl containing wet vermiculite. For spring wheat, the seedlings grew at 25° C. under a 12-h photoperiod for 7 days and then were transplanted into environmentally controlled greenhouse; for winter wheat, the plants grew at 25° C. under a 12-h photoperiod for 7 days and then were transferred to the 8° C. growth chamber for 30 days, afterwards the seedlings were transplanted into environmentally controlled greenhouse. The seeds were harvested individually according to each T₀ plant.

According to each T₀ plant, The seeds were harvested and soaked separately in 75 mg/L kanamycin solution (the amount is 1 seed can share 1 mL) for about 1.5 day at 25° C. until the seeds began to germinate, and then the seeds were sowed in the bowl containing wet vermiculite and placed at 25° C. under a 12-h photoperiod for 7 days. The green plants were counted and traced to their T₀ plants. Some parts of the leaves were collected from every green plant for detection. For winter wheat, the green plants were all transferred to the 8° C. growth chamber under a 12-h photoperiod for 30 days of vernalization, and then were transplanted into environmentally controlled greenhouse; for spring wheat, the green plants were directly transplanted into environmentally controlled greenhouse.

Total genomic DNA was extracted individually from the leaves of every plant. PCR was conducted with gus fragment primers: forward 5′-CAA CGA ACT GAA CTG GCA G-3′ and reverse 5′-CAT CAC CAC GCT TGG GTG-3′. Based on PCR results, the transformation degree was expressed as the percentage of the positive seeds for each individual T₀ plant; and the transformation rate was expressed as the percentage of T₀ plants which set positive seeds proved in T₁ generation.

(2) Results

Three genotypes of wheat, Jinhe 9123, China Spring and Bobwhite, were all successfully transformed.

Jinhe 9123: All 60 seeds germinated normally and were used for transformation. 50 of treated objects developed to plants and set seeds. 181 of T₁ green seedlings were obtained and they were from 29 T₀ plants. Based on the PCR results for all green plant, 77 of them were positive and they were from 17 T₀ plants. The transformation rate was 34% (17/50×100%).

\textcircled{2} Chinese Spring: All 30 seeds germinated normally and were used for transformation. 15 of treated objects developed to plants and set seeds. 59 of T₁ green seedlings were obtained and they were from 7 T₀ plants. Based on the PCR results for all green plant, 29 of them were positive and they were from 5 T₀ plants. The transformation rate was 33.3% (5/15 X 100%).

\textcircled{3} Bobwhite: All 30 seeds germinated normally and were used for transformation. 21 of treated objects developed to plants and set seeds. 47 of T₁ green seedlings were green and they were from 8 T₀ plants. Based on the PCR results for all green plant, 23 of them were positive and they were from 6 T₀ plants. The transformation rate was 28.6% (6/21×100%).

### Embodiment 3

### The Transformation for Apical Meristem of Rice Using the SMW Brush

(1) Materials and Methods

Rice cultivar: LongDao 10.

A. tumefaciens strain: EHA105.

The exogenous genes: gus gene and bar gene, constructed in vector pCAMBIA3301.

Single colony of A. tumefaciens was screened and inoculated into 50 mL of LB medium containing 50 mg/L kanamycin and 40 mg/L rifampicin, and grew to OD₆₀₀=0.6 at 28° C. on shaker with 220 rpm. The A. tumefaciens infection solution was obtained by centrifugating the culture at 4000 rpm for 5 min and re-suspended in the base buffer (1/2 volume of the original) containing 1/10 MS medium with 100 μM AS, 100 mg/L F68, 400 mg/L MES, 10 g/L glucose and 40 g/L maltose, pH 5.6.

120 full and complete seeds were screened and the hull was removed. The grains were sterilized routinely and placed on two layers of absorbent tissue in the Petri dish (Φ9 cm) containing 8 mL of sterilized water at 28° C. in dark for 1.5 days. 117 of them germinated normally. When their coleoptiles grew to about 0.2 cm, they were removed away by a tweezer to expose the apical meristem. The exposed meristem was stabbed and brushed for 2-3 times using SMW brush (200 bristles which are 8 μm in diameter for each, 0.5 mm in exposed length) dipped with the A. tumefaciens infection solution. Thereafter, the treated objects were placed in the autoclaved Petri dish (the inner tissue soaked with 1 mL of sterilized water) at 25° C. in dark for 3 days. Then the seedlings were planted into the environmentally controlled greenhouse and were covered with the straw mat which was sprayed water twice every day to keep the moisture. After 7 days, the straw mat was rolled up and the plants were managed normally until seeds matured. The seeds of the main spike and the tillering spikes were harvested and kept separately from each individual plant.

According to each T₀ plant, ten randomly selected seeds of each main spike were germinated separately and then sowed in the greenhouse. When the plants grow to one and a half leaves stage, a part of the leaf was collected and total genomic DNA were extracted from them. PCR was conducted with bar fragment primers: forward 5′-TCA AAT CTC GGT GAC GGG CA-3′ and reverse 5′-GGT CTG CAC CAT CGT CAA CC-3′. Based on PCR results, the transformation degree was expressed as the percentage of the positive seeds for each individual T₀ plant; and the transformation rate was expressed as the percentage of T₀ plants which set positive seeds proved in T₁ generation. If the seedlings of the ten seeds were all negative, PCR detection was conducted with all other seeds of the main spike.

(2) Results

117 out of 120 seeds germinated normally and were used for transformation. 77 objects grew to seedlings, thus the damage rate was 34.2%. 71 of them develop to set mature seeds. According to each T₀ plant, 10 randomly selected seeds of the main spike were germinated and sowed. When the plants grew to one and a half leaves stage, total genomic DNA was extracted for PCR analysis (FIG. 8). Results showed 153 out of 786 seedlings were positive and they were from 47 T₀ plants. It indicated that the transformation rate was 66.3% (47/71×100%). The transformation degree of these plants was 4.8% (1/21×100%) to 100% (10/10×100%), and the value is a kind of reference due to it is from ten seeds of main spike but not all seeds of the plant.

### Embodiment 4

The Transformation with BADH Gene for Apical Meristem of Rice Using SMW Brush

(1) Materials and Methods

Rice cultivar: ZhongHua 11.

A. tumefaciens strain: EHA105.

The exogenous genes: badh gene and npt II gene, constructed in vector pBIN438.

Single colony of A. tumefaciens was screened and inoculated into 50 mL of LB medium containing 50 mg/L kanamycin and 40 mg/L rifampicin, and grew to OD₆₀₀=0.5 at 28° C. on shaker with 220 rpm. The A. tumefaciens infection solution was obtained by centrifugating the culture at 4000 rpm for 5 min and re-suspended in the base buffer (1/4 volume of the original) containing 1/10 MS medium complemented with 100 μM AS, 100 mg/L F68, 400 mg/L MES, 10 g/L glucose and 40 g/L maltose, pH 5.6.

30 full and complete seeds were screened and the hull was removed away. The grains were sterilized routinely and placed on two layers of absorbent tissue in the Petri dish (Φ9 cm) containing 8 mL of sterilized water at 28° C. in dark for 1.5 days. All of them germinated normally. When the coleoptile grew to approximately 0.2 cm, it was removed away by a tweezer to expose the apical meristem. The exposed meristem was stabbed and brushed for 2-3 times using SMW brush (300 bristles which are 8 μm in diameter for each, 1 mm in exposed length) dipped with the A. tumefaciens infection solution. Thereafter, the treated objects were placed in the autoclaved Petri dish (the inner tissue soaked with 1 mL of sterilized water) at 25° C. in dark for 3 days. Then the seedlings were planted into the environmentally controlled greenhouse and covered with plastic film for 10 days and then were managed normally until seeds matured. The seeds were harvested from each individual plant and stored separately. According to each T₀ plant, the seeds were geminated and sowed separately in the greenhouse. When the plants grew to one and a half leaves stage, a part of the leaf was collected and total genomic DNA were extracted from them. PCR was conducted with badh fragment primers: forward 5′-ATT GGC ATC TGT GAC TT-3′ and reverse 5′-CAC TCG CTT GAC TCC TTC-3′. Based on the PCR results, the transformation degree was expressed as the percentage of the positive seeds for each individual T₀ plant; and the transformation rate was expressed as the percentage of T₀ plants which set positive seeds proved in T₁ generation.

(2) Results

All of 10 seeds germinated normally and were used for transformation. 7 of them grew to seedlings and 4 of them set seeds. According to each T₀ plant, all seeds were harvested and sowed separately, 91 T₁ seedlings were obtained. When the plants grew to one and a half leaves stage, total genomic DNA was extracted from a part of leaf of individual plant. PCR results showed that 9 of the seedlings were positive and they were from 2 T₀ plants. It indicated that the transformation rate was 50% (2/4×100%). The transformation degree: for line 3 was 50%, in which 12 seedlings were detected by PCR and 6 of them were positive (6/12×100%); and for line 4 was 25%, in which 12 seedlings were detected by PCR and 3 of them were positive (3/12×100%) respectively.

### Embodiment 5

### The Transformation for Apical Meristem of Maize Using SMW Brush

(1) Materials and Methods

Transformation object: HY489 inbred line.

A. tumefaciens strain: EHA105.

The exogenous genes: bar gene, bt gene and pta gene, constructed in vector pCAMBIA3300.

Single colony of A. tumefaciens was screened and inoculated into 50 mL of LB medium containing 50 mg/L kanamycin and 40 mg/L rifampicin, and grew to OD₆₀₀=0.6 at 28° C. on shaker with 220 rpm. The A. tumefaciens infection solution was obtained by centrifugating the culture at 4000 rpm for 5 min and re-suspended in the base buffer (1/2 volume of the original) containing 1/10 MS medium complemented with 100 μM AS, 100 mg/L F68, 400 mg/L MES, 10 g/L glucose and 40 g/L maltose, pH 5.6.

20 normal and healthy seeds were sterilized with 20 mL of sodium hypochlorite (2.0%) for 20 min and rinsed five times in sterilized water. Then the seeds were placed on two layers of absorbent tissue dripped with 13 mL of sterilized water in the Petri dish (Φ9 cm) at 28° C. in dark for 2 days. 15 of them germinated normally. When the coleoptiles grew to 0.3-0.6 cm, they were removed away to expose the apical meristem. Then the exposed meristem was stabbed and brushed for 2-3 times using SMW brush (2000 bristles which are 20 μm in diameter for each, 3 mm in exposed length) dipped with the A. tumefaciens infection solution. Thereafter, the seeds were placed in the autoclaved Petri dish with two layers of absorbent tissue dripped with 3 mL of sterilized water. The Petri dish was covered with lid and placed at 25° C. in dark for 3 days. Afterwards, the seedlings were planted to the bowl containing wet vermiculite at 25° C. under natural light for 7 days, and then they were transplanted into the environmentally controlled greenhouse. Both the tassel and ear were protected with bags at suitable time and artificial pollination was carried out to set selfing seeds.

According to each T₀ plant, the seeds were harvested and then germinated separately. Total genomic DNA was extracted from the leaves of every T₁ plant for PCR detection. Based on the PCR results, the transformation rate and transformation degree were calculated. Some positive plants were screened and send to Beijing Meilaibo Medical Technology Co. Ltd for Southern blot analysis.

(2) Results

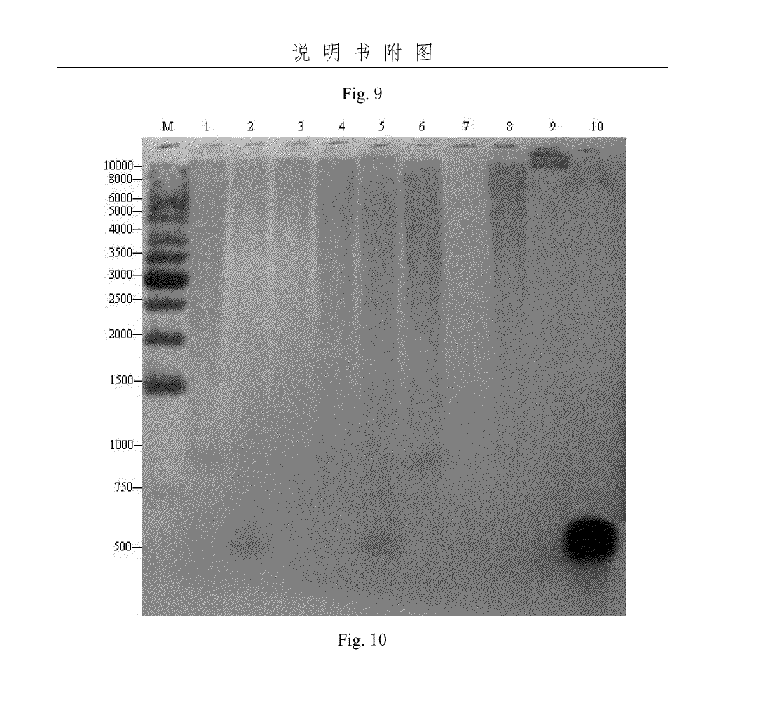

15 out of 20 seeds germinated normally and were used for transformation. 9 of them grew to seedlings and 6 of them set seeds. Seeds of these 6 plants were screened (20 seeds for each) and germinated to seedlings (T₁). PCR results showed that 39 T₁ plants were positive and they were from 5 T₀ plants (FIG. 9). For the line 3, no positive plant was found in the first 20 seeds, but 7 plants were positive in the following detection with other 36 seeds. It showed that positive seeds were set in every T₀ plant, the transformation rate was 100%. The reference value of transformation degree for each T₀ plant was 5-70% (details were shown in table below). Southern blot indicated that single copy of exogenous gene was integrated (FIG. 10).

The reference value of transformation
degree (R-transformation degree)

| T₀ line | 1 | 2 | 3 | 4 | 5 | 6 |
|---|---|---|---|---|---|---|
| Detected plants (T₁) | 20 | 20 | 20 + 36 | 20 | 20 | 20 |
| Positive plants (T₁) | 14 | 1 | 7 | 5 | 10 | 9 |
| R-transformation degree(%) | 70 | 5 | 12.5 | 25 | 50 | 45 |

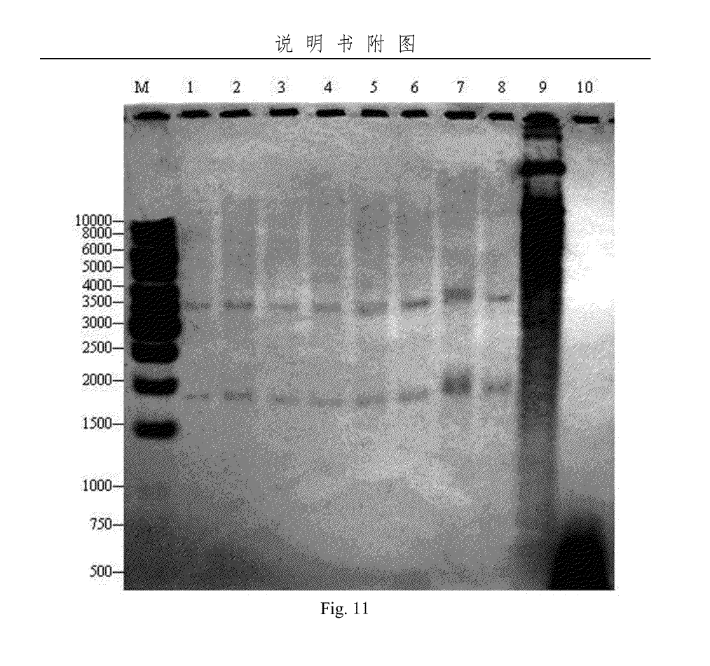

Additionally, in the 26^{th} batch of maize transformation, Southern blot results showed that the exogenous gene was two copy integrations with the genomic DNA of T₂ plants (FIG. 11).

### Embodiment 6

### The Transformation for Apical Meristems of Millet, Broomcorn Millet and Sorghum Using the SMW Brush

(1) Materials and Methods

Transformation objects: a millet with yellow hull (yellow millet), a millet with red hull (red millet), a broomcorn millet with red hull (red broomcorn millet), a broomcorn millet with white hull (white broomcorn millet), and a sorghum.

A. tumefaciens strain: EHA105.

The exogenous genes: gus gene and npt-II gene, constructed in vector pCAMBIA2201.

Single colony of A. tumefaciens was screened and inoculated into 50 mL of LB medium containing 50 mg/L kanamycin and 40 mg/L rifampicin, and grew to OD₆₀₀=0.6 at 28° C. on shaker with 220 rpm. The A. tumefaciens infection solution was obtained by centrifugating the culture at 4000 rpm for 5 min and re-suspended in the base buffer (1/3 volume of the original) containing 1/10 MS medium complemented with 100 μM AS, 100 mg/L F68, 400 mg/L MES, 10 g/L glucose and 40 g/L maltose, pH 5.6.

Full and complete seeds of yellow millet, red millet, red broomcorn millet, white broomcorn millet, and sorghum were screened and the hull was removed away. The grains were sterilized routinely and placed in the autoclaved Petri dish (Φ9 cm) containing two layers of absorbent tissue dripped with 8 mL of sterilized water at 28° C. in dark for about 1.5 days. When the coleoptile grew to 0.1-0.2 cm (the subterranean stem have elongated, and the length was: millet>broomcorn millet>sorghum), the coleoptile and little leaves were cut away by a blade just in the above of the refraction belt between the subterranean stem and the coleoptile, in which it is the region of the apical meristem located. The exposed meristem was stabbed and brushed for 2-3 times using SMW brush dipped with the A. tumefaciens infection solution (the specification of brush for the transformation of millet: 90 bristles which are 4 μm in diameter for each, 0.5 mm in exposed length; for broomcorn millet: 100 bristles which are 8 μm in diameter for each, 1 mm in exposed length; for sorghum: 300 bristles which are 10 μm in diameter for each, 1.5 mm in exposed length). Thereafter, the treated objects were placed in the autoclaved Petri dish (Φ9 cm) containing two layers of absorbent tissue dripped with 1 mL of sterilized water at 25° C. in dark for 3 days. Afterwards, the seedlings were planted into the environmentally controlled greenhouse and covered with the plastic film for 10 days and then were managed normally until seeds matured.

Seeds were separately harvested from each T₀ plant, and the 1/10 of the total seeds of the ear (or main ear) top was firstly harvested for detection. 10 of them randomly selected were germinated and then sowed in the greenhouse. When the plants grew to two and a half leaves stage, total genomic DNA was extracted from the leaves of each T₁ plant for PCR using gus fragment primers: forward 5′-CAA CGA ACT GAA CTG GCA G-3′ and reverse 5′-CAT CAC CAC GCT TGG GTG-3′. Based on the PCR results, the transformation rate was expressed as the percentage of T₀ plants which set positive seeds proved in T₁ generation.

(2) Results

Positive plants were obtained by sufficient and micro wounding transformations for apical meristems of all the five cultivars from these three species. It indicated that this method has a wide applicability. The details were showed below:

\textcircled{1} Yellow millet: 17 germinated seeds were used for transformation, 13 of them developed normally and set seeds. Based on PCR results in T₁ seedlings, positive plants were found in 5 T₀ plants and the transformation rate was 38.5% (5/13×100%).

\textcircled{2} Red millet: 31 germinated seeds were used for transformation, 29 of them developed normally and set seeds. Based on PCR results in T₁ seedlings, positive plants were found in 15 T₀ plants and the transformation rate was 51.7% (15/29×100%).

\textcircled{3} Red broomcorn millet: 22 germinated seeds were used for transformation, 20 of them developed normally and set seeds. Based on PCR results in T₁ seedlings, positive plants were found in 13 T₀ plants and the transformation rate was 65.0% (13/20×100%).

White broomcorn millet: 42 germinated seeds were used for transformation, 40 of them developed normally and set seeds. Based on PCR results in T₁ seedlings, positive plants were found in 19 T₀ plants and the transformation rate was 47.5% (19/40×100%). Sorghum: 17 germinated seeds were used for transformation, 15 of them developed normally and set seeds. Based on PCR results in T₁ seedlings, positive plants were found in 11 T₀ plants and the transformation rate was 73.3% (11/15×100%).

1. A method of shoot apical meristem transformation for monocot plant via sufficient and micro wounding (SMW), comprising the steps of:
(1) Preparation of in vivo meristems and infection solution
Select healthy and complete seeds of objective crops, removing the chaff or the husk away; wash the seeds clean and soak them in water at 25° C. for 7-10 hours; after routine sterilization, rinsing the seeds in sterilized water several times and placing them on two layers of autoclaved absorbent tissue in a Petri dish (Φ90 mm); dripping sterilized water with an amount just to keep the absorbent tissue wet, and then germinating the seeds at 28° C. in dark for 1-2 days; said in vivo meristem for transformation is the shoot growing point of the seed treated in this way;
Screen single colony of A. tumefaciens containing binary vector harboring exogenous genes, and inoculating it into LB medium containing 50 mg/L kanamycin and 40 mg/L rifampicin and growing to OD₆₀₀=0.5-0.6 at 28° C. on shaker with 220 rpm in dark; preparing the A. tumefaciens infection solution by centrifugating the culture at 4000 rpm for 5 min and re-suspending it in the base buffer of 1/5-1/2 volume as the original; said base buffer contains 1/10 MS medium supplemented with 100 μM AS, 100 mg/L F68, 400 mg/L IVIES, 10 g/L glucose and 40 g/L maltose, pH 5.6;
(2) Expose the shoot apical meristem and transform it via SMW
\textcircled{1} Initial time management: conducting the transformation as early as possible, wherein the suitable time is when the shoot has grown to 0.2-2 cm for little grain plant, and 0.3-1 cm for big grain plant;
Said little grain plants comprise wheat, rice, millet, broomcorn millet, and sorghum; said big grain plant comprises maize;
\textcircled{2} Method to expose the shoot apical meristem
For the plants in which subterranean stem has not elongated, directly removing the coleoptile and little leaves away from its base using a tweezer; for the plants in which subterranean stem have elongated, cutting the coleoptile and little leaves away using a blade just above of the light reflection belt between the subterranean stem and the coleoptile, where it is the region of the shoot apical meristem located;
\textcircled{3} Transformation using the SMW brush
stabbing and brushite the shoot apical meristem for 2-3 times using the SMW brush dipped with the A. tumefaciens infection solution; thereafter, place the seeds with shoot apical meristem up on two layers of dry absorbent tissue in the Petri dish which have been autoclaved together; each Petri dish can be put in 10-40 seeds;
(3) Co-cultivation
Dripping 0.5-3 mL of sterilized water on the absorbent tissue in the Petri dish containing transformation treated seeds, and then specially co-culturing the in vivo meristems at 25 ° C. in dark for 3 days with the dish lid covered;
(4) Develop to seedlings
After co-cultivation, cover the roots with vermiculite in the dish, or transplanting the little seedlings to a bowl containing vermiculite;
For the plants which do not require vernalization, grow the little seedlings at 25° C. with a 12-h photoperiod for 7 days, and then transplanting them into pot in greenhouse, or directly transplanting the young seedlings into pot in greenhouse if they have developed relatively big and cover with plastic film to recover and protect them for 7-10 days;
For the plants which require vernalization, such as winter wheat, growing the little seedlings at 25° C. with a 12-h photoperiod for 7 days, and then transferring them into the 8° C. growth chamber for 20-30 days, the time required depends on the cultivar;
(5) Transplant the seedlings
Transplanting the seedlings into the environmentally controlled greenhouse or farmland, when they are developed enough;
(6) Seedling and plant management
Promoting the seedlings and plants healthily to develop big spikes and set more seeds with water and nutrition management, for maize with unisexual flowers, protect the ear and tassel with paper bags and then carry out the pollination artificially;
(7) Molecular detection and identification
Do not perform the detection, selection and identification in T₀ plants to avoid false results; harvest the seeds of T₀ plants separately; perform the detection and identification in T₁ generation; for the plants which have been transformed with exogenous vector harboring resistance gene, screen the resistant plants and then carry out PCR identification; for the plants transformed without resistance gene, carry out PCR identification directly; perform Southern blot analysis for PCR-positive plants,
wherein the bristles of said SMW brush are made of stainless steel fibers, glass fibers or carbon silicon fibers in micron-grade, one bristle is 4-20 μm in diameter and 0.5-3 mm in exposed length, and one brush contains 100-5000 bristles.

2. (canceled)

3. The method of claim 1, wherein said SMW brush in which bristle is 8-18 μm in diameter, the bristle is 1-2 mm in exposed length, and each brush contains 100-2000 of bristles.

4. The method of claim 1, wherein said “stabbing and brushing” comprises stabbing and brushing on the shoot apical meristem, said “stabbing” is to prick the apical meristem vertically with the SMW brush dipped with the A. tumefaciens infection solution to transfer the exogenous genes; and said “brushing” is to comb the whole apical meristem with the SMW brush dipped with the A. tumefaciens infection solution to transfer the exogenous genes.

5. The method of claim 4, wherein said plants which subterranean stem could not elongate comprise wheat and rice; said plants which subterranean stem could elongate comprise maize, millet, broomcorn millet, and sorghum.


SEQUENCE LISTING (6 sequences)

SEQ ID NO: 1 - DNA, 19 bp, Artificial Sequence
  Gus fragment primer
caacgaactgaactggcag

SEQ ID NO: 2 - DNA, 18 bp, Artificial Sequence
  Gus fragment primer
catcaccacgcttgggtg

SEQ ID NO: 3 - DNA, 20 bp, Artificial Sequence
  Bar fragment primer
tcaaatctcggtgacgggca

SEQ ID NO: 4 - DNA, 20 bp, Artificial Sequence
  Bar fragment primer
ggtctgcaccatcgtcaacc

SEQ ID NO: 5 - DNA, 17 bp, Artificial Sequence
  Badh fragment primer
attggcatctgtgactt

SEQ ID NO: 6 - DNA, 18 bp, Artificial Sequence
  Badh fragment primer
cactcgcttgactccttc